(19) United States
(12) Patent Application Publication
(10) Pub. No.: US 2020/0082933 A1
(43) Pub. Date: Mar. 12, 2020

(54) PRE-AUTHORIZATION PROCESS USING BLOCKCHAIN

(71) Applicant: INTERNATIONAL BUSINESS MACHINES CORPORATION, ARMONK, NY (US)

(72) Inventors: Fang Lu, Billerica, MA (US); Uri Kartoun, Cambridge, MA (US); Shilpa N. Mahatma, Chappaqua, NY (US); Vishrawas Gopalakrishnan, Cambridge, MA (US)

(21) Appl. No.: 16/124,318

(22) Filed: Sep. 7, 2018

(51) Int. Cl. G16H 40/20; H04L 9/06; G16H 10/60

(52) U.S. Cl. CPC G16H 40/20; G16H 10/60; H04L 9/0637

(57) ABSTRACT

A method, computer system, and a computer program product for pre-authorization is provided. The present invention may include identifying a treatment for a medical condition that yields a positive outcome. The present invention may also include identifying a plurality of key features in the identified treatment. The present invention may then include identifying a plurality of features within the plurality of key features that can be applied to a patient. The present invention may further include identifying a stakeholder based on the identified plurality of features applied to the patient. The present invention may also include authorizing the identified stakeholder. The present invention may then include creating a new block based on the authorized stakeholder. The present invention may further include adding the new block to a blockchain network for processing authorizations.

### BACKGROUND

The present invention relates generally to the field of computing, and more particularly to the healthcare industry. Current government regulations and medical insurance policies are complicated and take a significant amount of time for a patient to get approved to see a specialist, get a second opinion or get a special test or procedure done. In the time it takes to get approval, the health condition of a patient may have deteriorated, and the delay may also result in life threatening medical conditions.

### SUMMARY

Embodiments of the present invention disclose a method, computer system, and a computer program product for pre-authorization. The present invention may include identifying a treatment for a medical condition that yields a positive outcome. The present invention may also include identifying a plurality of key features in the identified treatment. The present invention may then include identifying a plurality of features within the plurality of key features that can be applied to a patient. The present invention may further include identifying a stakeholder based on the identified plurality of features applied to the patient. The present invention may also include authorizing the identified stakeholder. The present invention may then include creating a new block based on the authorized stakeholder. The present invention may further include adding the new block to a blockchain network for processing authorizations.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for pre-authorization using blockchain according to at least one embodiment;

FIG. 3 is a block diagram of internal and external components of computers and servers depicted in FIG. 1 according to at least one embodiment;

FIG. 4 is a block diagram of an illustrative cloud computing environment including the computer system depicted in FIG. 1, in accordance with an embodiment of the present disclosure; and

FIG. 5 is a block diagram of functional layers of the illustrative cloud computing environment of FIG. 4, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for pre-authorization. As such, the present embodiment has the capacity to improve computer capabilities in, for example, the medical field associated with the pre-authorization process by identifying stakeholders (i.e., shareholders or approvers) who may authorize treatments to reduce the time it takes for a patient to see a specialist. More specifically, stakeholders may be dynamically searched for, identified and consolidated based on a patient medical condition, positive outcomes for the medical condition and an insurance policy or governmental guidelines for treatments. Natural language processing (NLP), machine learning (ML) and semantic analysis may be used to create the dynamic identification and consolidation of stakeholders to reduce patients wait times, provide patients with quicker access to medical care, improve patient recovery times and overall well-being. Once the stakeholders are searched, identified and consolidated, the stakeholders are introduced (i.e., appended) to the block in a blockchain network for a secure and transparent pre-authorization process.

As previously described, current government regulations and medical insurance policies are complicated and take a significant amount of time for a patient to get approved to see a specialist, get a second opinion or get a special test or procedure done. In the time it takes to get approval, the health condition of a patient may have deteriorated, and the delay may also result in life threatening medical conditions. Average wait times to see a specialist can last one month to two months and in higher specialized areas of the medical industry (e.g., neurosurgery), wait times can be three months.

A current patient pre-authorization process in neurosurgery, for example, the patient will need to obtain preoperative medical clearance. Preoperative medical clearance may require the primary care physician (e.g., internist or cardiologist) of the patient to obtain and check blood tests and pressure, urine tests, an electrocardiogram and a chest x-ray. The preoperative clearance procedure is done to ensure the patient is medically healthy enough to recover from surgery. For example, checking the blood test and pressure of a patient will provide information that the blood pressure is well-controlled and blood sugar is within healthy ranges to withstand surgery. Preoperative testing may be done 7-10 days prior to the neurosurgery.

Some patients may require additional testing before medical clearance is granted for surgery. For example, a patient with a history of heart disease may require an echocardiogram or a stress test in addition to the standard electrocardiogram. The patient has the responsibility to contact health insurance providers for pre-authorization prior to the scheduling of preoperative doctor visits, exams, labs and other tests prior to surgery and the current pre-authorization process may significantly slow the process down from the time the patient needs surgery to the time the patient actually gets the needed surgery.

Government regulations and insurance policies can be complicated and may also play a significant role in the extended wait times. Therefore, it may be advantageous to, among other things, create a system that analyzes patient medical conditions, locations, insurance policies and other guidelines to find stakeholders that may authorize treatment by using natural language processing (NLP) and machine learning (ML). A stakeholder may be determined based on the current medical condition of the patient, treatments available for the medical condition and the required parties available on the medical insurance policy or a governmental regulation. For example, some insurance policies require a primary care physician to prescribe medicine or pre-approve the patient to see a specialist while other insurance policies do not have the same requirements. An additional example may include government regulations for certain medical treatments that require additional steps or approvals for the prescribed treatments. Stakeholders may include, for example, an insurance representative, one or more doctors, a hospital administrator, a government employee, a pharmaceutical company, family members of the patient (e.g., if a minor, incapacitated or in a coma) or a healthcare proxy.

According to at least one embodiment, in the medical field, a pre-authorization system may be integrated with patient electronic health records (EHRs), the health insurance policy rules stored on a database and a central government healthcare regulation database. The pre-authorization system may work cohesively with existing technologies and use the output data to extract key features that may be used to analyze and determine a patient treatment plan that may yield a positive medical outcome, result or conclusion. One existing technology output data may include a patient similarity analytics that identifies similar patients (i.e., cohorts) with similar features as the patient. One other existing technology output data may include IBM® CareFlow (IBM CareFlow and all IBM CareFlow-based trademarks and logos are trademarks or registered trademarks of International Business Machines Corporation and/or its affiliates). IBM® CareFlow may assist both doctors and patients to create a healthcare plan that may provide better outcomes by observing the most effective historical outcomes of other similar patients.

Key features (i.e., features, extracted features or critical features) in the medical domain may include factors, such as, patient treatment plans that yield positive results, specialist visit procedures, special lab tests, physical location, insurance policy, accessibility to medical facilities, medical condition, physical condition, age, gender, race, existing family history, use of tobacco, use of alcohol, profession, prescribed medication, lab results, examination procedures and results, blood test results, computed tomography (CT) scan results and magnetic resonance imaging (MRI) results. Extracted features may be obtained from stored data on one or more databases, knowledgebases or corpora. The stored data may include, for example, data stored on a medical facility database, a national medical database, an insurance company database, a government database or a database that stores patient EHRs.

The pre-authorization system may use natural language processing (NLP) to extract key features, such as word embedding techniques that map words and phrases to vectors of numbers using mathematical embedding. When words and phrase embeddings are used, the NLP accuracy improves semantic analysis. Word embedding techniques incorporated with the pre-authorization system may identify the closest features that may be applied to the current patient.

For example, in the medical domain, the NLP algorithms will use data available in the patient EMR files as an input source. The EMR files may be scanned for data, such as pre-defined keywords that may be used for each type of surgery use case. Pre-defined keywords may be entered manually or retrieved from machine learning rules scanning, for example, a medical ontology database. NLP algorithms may extract, for example, relevant prescription medication information, lab results, examination results, procedures or surgery information based on the medical treatment the patient requires. Additionally, the NLP algorithm may further associate the corresponding stakeholders for each step, such as the internal medicine doctor, the radiologist and the neurosurgeon.

Semantic analysis may be used to infer the meaning and intent of the words and phrases, both verbal and non-verbal. For example, verbal meaning may be inferred using the spoken word and phrases captured by a microphone from a doctor verbally inputting and storing notes on a medical database (i.e., corpus or knowledgebase) and nonverbal meaning may be inferred using words or phrases captured in stored documents on a medical database. Semantic analysis may consider current and historical activities associated with a patient and with cohorts of the patient, for example if the patient is suffering from a particular symptom, other patients with similar symptoms and ailments whom have had successful treatments may be analyzed and may be useful for the current patient. Semantic analysis may also consider syntactic structures at various levels to infer meaning to words, phrases, sentences and paragraphs.

Machine learning (ML) may be incorporated by, for example, analyzing insurance policy data, hospital guidelines and government guidelines for a specified treatment for a patient at a particular hospital location or within a particular hospital network. The ML algorithm may be used to analyze existing patients (i.e., cohorts of the patient) who have undergone similar surgeries by considering various recovery results, insurance policy data, hospital guidelines and government guidelines for a specified treatment plan for a patient at a particular hospital location or within a particular hospital network. The ML algorithm may determine what the critical factors are that will generate the most optimal patient recovery results. The critical factors may be determined, for example, based on the recovery results of the existing patients, the health insurance covered doctors' visits, lab results, examination results, surgery results and permitted government healthcare guidelines.

Once features have been extracted, the features may be converted and associated with stakeholders, for example, based on the patient's physical location, insurance policy and accessibility to medical facilities. The patient's current health insurance policy may be examined, and features may be extracted to analyze which doctors may be available in the network and which types of coverage may be allowed for specific procedures, labs or additional tests. The current medical and physical conditions of the patient may be analyzed with regards to the patient's ability to get to a suggested medical facility (e.g., located in an area the patient can drive to).

Stakeholders may be identified and determined based on the patient data, such as medications, lab results, examination results, and surgeries that may be required for the full treatment plan for the patient within the guidelines of the patient insurance policy. The pre-authorization system may run keyword searches, for example, in the insurance policy for each medication and procedure needed by the patient to validate (i.e., check, identify, verify or determine) that each medication and procedure are covered in the health insurance policy.

If a patient is traveling, then the location of the patient may be determined, for example, by the patient's smart phone, smart watch, tablet or computing device that has global position system (GPS) capabilities. Alternatively, the pre-authorization system may send an inquiry (e.g., email message, text message, popup notification or automated voice message) to the patient to ask the patient to report a current physical location for treatment to determine medical coverage is within the medical network. For example, if the patient travels outside of the current medical network, such as a different state, then the patient may need to seek a doctor other than the in-network primary care physician or may need to see a different specialist. The alternate internal medicine doctor or the specialist may be advised that the new location of the patient may not provide coverage to the patient in the different state and in a potentially different health insurance network.

The pre-authorization system may then identify and introduce the stakeholders into the current patient healthcare blockchain process for pre-authorization. Blockchain technology may be used for a network system to have consensus, authenticity, traceability, transparency, immutability and finality of logged data (i.e., events, transactions or authorizations). Blockchain technology may consist of a shared log of events that may be kept in blocks of data that are passed to the next transaction in linear order. Thereafter, the transaction (i.e., pre-authorization and authorization) may be noted in the ledger. For example, Pre-Authorization Party A is the patient's primary care physician, Pre-Authorization Party B is a medical specialist and Pre-Authorization Party C is the insurance company. Each party, A, B and C, may need to provide authorization and consensus in order for the patient to seek medical care from the medical specialist (i.e., Pre-Authorization Party B). Once a consensus is reached, the block may be appended to the blockchain and the transaction may be noted in the ledger.

If the ledger had an inconsistency or the record was destroyed, then recovery of the transaction details may be very difficult. With blockchain technology, when a transaction is written into a ledger, there may be a mechanism to make sure all the records are synchronized and if the network system detects an error, the error may be immediately corrected. Blockchain systems may be immutable since the system creates a hash value on each block and any change in data will show a different hash value, which may increase the security and transparency of each transaction by not allowing transactions to be removed or deleted. Pre-authorization transaction requests and responses may not be tampered with on a blockchain system which may reduce errors and increase the security of the pre-authorization process.

A pre-authorization system running on blockchain allows pre-authorization requests and authorization decisions to be traceable (i.e., can trace the time, date and from whom the request and decision was made). The tracible medium (i.e., blockchain) is efficient since many requests, decisions and authorizations may be initiated and decided by incorporating, for example, various different institutions, rules and regulations, test results, patients and doctors. The traceability of the data may additionally be used to increase accountability and reduce patient wait times (i.e., higher efficiency) by each institution and each person with authority since the institution and authoritative figure may be privy to the transparency of the data. For example, a hospital administrator may have access to the blockchain network that stores the pre-authorization and authorization data. The hospital administrator may view how long it took a doctor in the network to approve a pre-authorization request.

When a request for pre-authorization is initiated, a block is created for the stakeholders. The corresponding stakeholders may be identified when the critical features have been concluded based on the analysis from cohorts that may share similar attributes with the current treated patient. When the pre-authorization process is initiated (i.e., initialized), the authorization requests may be transmitted, for example, via an email message, a text message, a popup notification or an automated voice message to each stakeholder for parallel processing. Parallel processing may allow simultaneous execution of computer operation processes that provides stakeholders with the ability to quickly approve a pre-authorization and allows each stakeholder approval to be independent of each other stakeholder approval.

The pre-authorization system may track a stakeholder response time (e.g., time taken by shareholder to approve a pre-authorization request) by calculating the time duration between when the pre-authorization request was initialized to the time the request was completed by the shareholder. Each stakeholder may be issued, for example, a user identification (i.e. user ID) and a password when the stakeholder is identified and added to the blockchain system. The pre-authorization system may track the approval time (i.e., how long the stakeholder takes to respond to the pre-approval request) and save the approval time as records. The stakeholders may use the pre-authorization system user ID and password to login and view the data available to respond to a pre-authorization request and the data that shows the approval duration time. A request approval report and the duration time for the response to the request may be shared by individual stakeholders and relevant parties as needed with permission.

A new block may be generated once the authorizations are done for the additional stakeholders. Transaction data (i.e., authorizations, requests or events) may be permanently recorded into files called blocks. Blocks may be considered, for example, individual pages of a city recorder's record book that holds the records of changes to title in real estate transactions. Another example of a block may include a stock transaction ledger. Blocks may be organized into a linear sequence over time, creating a blockchain.

Then, the new block may be appended to the blockchain for processing. New transactions may be continually processed by miners (i.e., miner nodes) into new blocks which may be added to the end of the chain. A miner may have the ability to create blocks using the transaction (i.e., event or pre-authorization request) and by adding the event to the transaction records, for example, to a public ledger of past transactions. A mining ring may be considered, for example, a single computer system that may perform necessary computations for mining.

In an alternate embodiment, the pre-authorization system may use the blockchain network with a smart contract. The smart contract may know who the authorizers are when the smart contract is setup. For example, if conditions A, B and C are met, then D needs to authorize the transaction, event or medical treatment. D may automatically authorize the transaction given that there is criticality to the authorization request. Additionally, a manual decision (i.e., request for authorization or pre-authorization) may be recorded in the blockchain and a smart contract may be applied to an automatic pre-authorization based on the profile of the individual or the patient. The information provided manually or provided or received from the smart contract may use ML to record the requests or decisions and the reasons why the requests or decisions were made. In the event that an authorizer does not respond quickly, depending on the criticality of the authorization request, the smart contract may enable backup authorizers to ensure fast responses.

In a second alternate embodiment, the pre-authorization system may be used in industries other than healthcare, such as automotive, private business, government, agriculture, shipping and industrial. Fields that use insurance policies or other complicated approval rules may also benefit from the pre-authorization system.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language, python programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

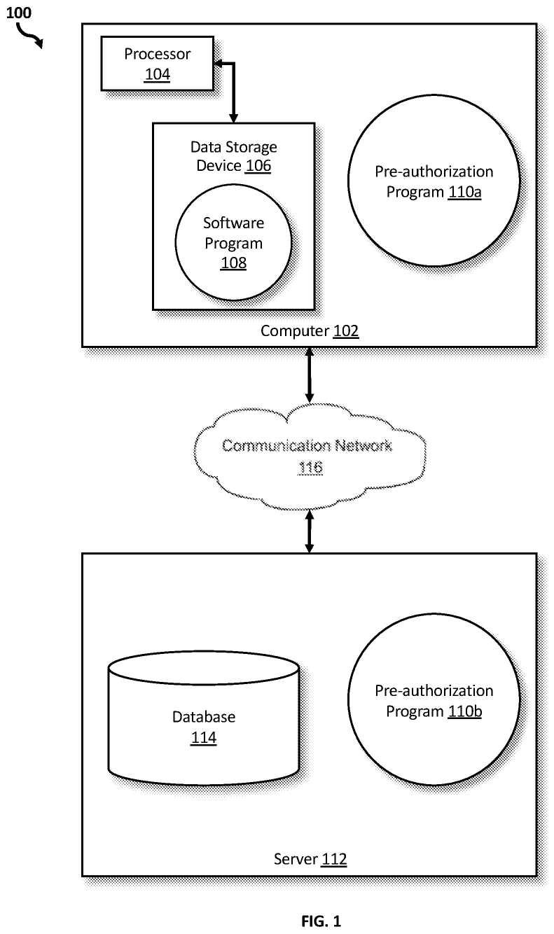

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a pre-authorization program 110a. The networked computer environment 100 may also include a server 112 that is enabled to run a pre-authorization program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

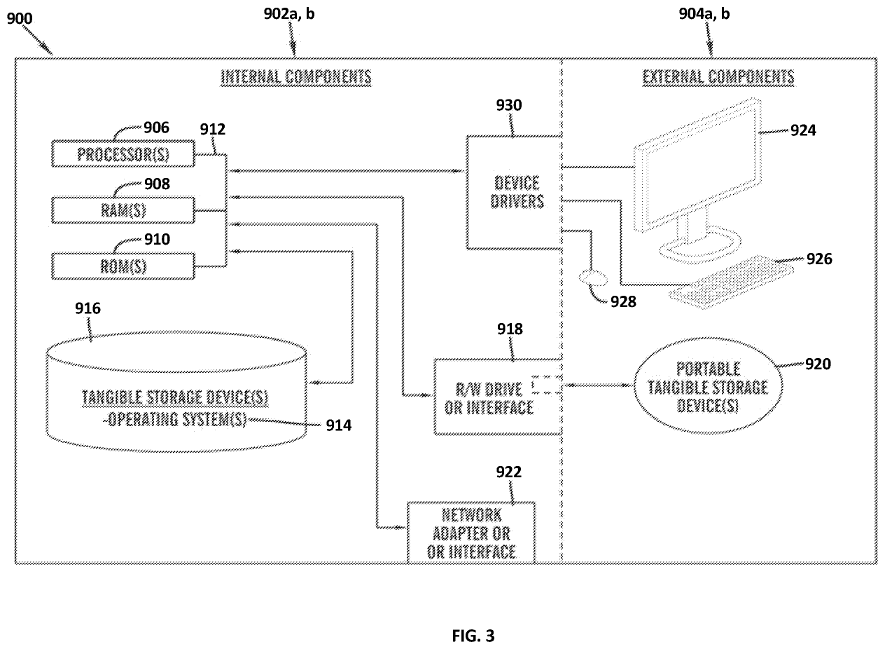

The client computer 102 may communicate with the server computer 112 via the communications network 116. The communications network 116 may include connections, such as wire, wireless communication links, or fiber optic cables. As will be discussed with reference to FIG. 3, server computer 112 may include internal components 902a and external components 904a, respectively, and client computer 102 may include internal components 902b and external components 904b, respectively. Server computer 112 may also operate in a cloud computing service model, such as Software as a Service (SaaS), Analytics as a Service (AaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the pre-authorization program 110a, 110b may interact with a database 114 that may be embedded in various storage devices, such as, but not limited to a computer/mobile device 102, a networked server 112, or a cloud storage service.

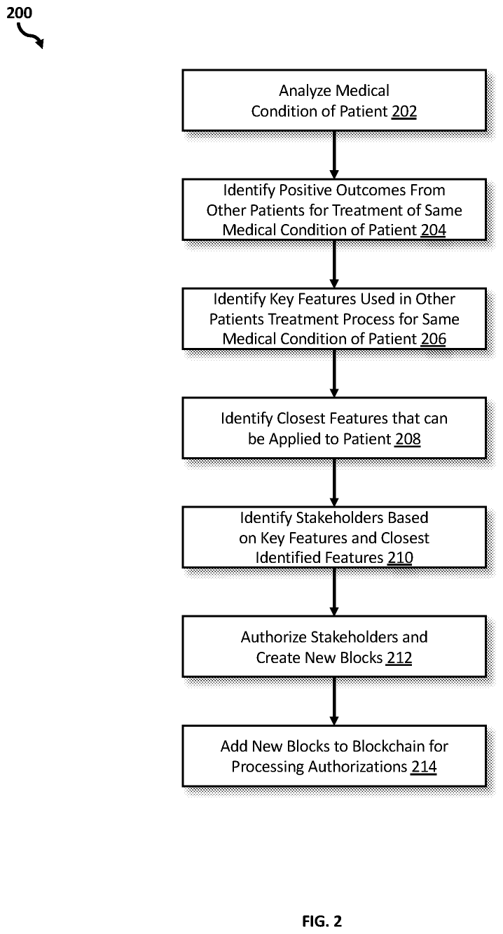

According to the present embodiment, a user using a client computer 102 or a server computer 112 may use the pre-authorization program 110a, 110b (respectively) to reduce patient referral wait times. The pre-authorization method is explained in more detail below with respect to FIG. 2.

Referring now to FIG. 2, an operational flowchart illustrating the exemplary pre-authorization process 200 used by the pre-authorization program 110a, 110b according to at least one embodiment is depicted.

At 202, a medical condition of a patient is analyzed. The medical condition of the patient may be, for example, entered and stored into an EHR database. NLP may be used, for example, to parse, tag and analyze words in the EHR database related to the key features of the medical condition of the patient. NLP algorithms may be used to analyze features, such as the patient medical condition, symptoms, diseases, recommended surgery, age, family medical history, location to medical facilities and physical condition. A patient may require medical treatment other than basic medical care. For example, a patient may have a symptom that is new, and the new symptom requires a medical specialist or an unconventional medical test prior to surgery. Pre-defined keywords may be used, for example, in the NLP algorithm for the type of surgery that is needed (e.g., keywords from a brain surgery ontology database) based on the patient treatment plan. NLP algorithms may also extract procedures, medication and examination information relevant to the surgery.

For example, a patient visits the ER in an ambulance due to a stroke. The patient is admitted to the hospital and the medical condition is entered into the hospital database. Following the hospital visit, the doctor determines that the patient needs surgery to repair a defective blood vessel. The patent data (e.g., features) are now available in the patient EHR database and the patient data may be used by the pre-authorization program 110a, 110b to begin the determination process of analyzing which pre-authorizations are needed leading up to the surgery and during the recovery process.

Then, at 204, positive outcomes from other patients (i.e., cohorts) for the treatment of the same medical condition of the patient are identified. Medical diagnoses, treatments and results may be stored on various databases and patient EHRs. The databases may be searched using NLP and semantic analysis to parse the content of the databases to identify specific medical conditions (e.g., symptoms), how they conditions were treated and if the treatment was a success. For example, the pre-authorization program 110a, 110b evaluates the treatment for patients who have visited the emergency room (ER) due to a sudden severe headache. The pre-authorization program 110a, 110b will check the medications, lab test results and the CT or MRI exam results that were taken by the patients. A positive outcome may be determined, analyzed and identified if an ER patient was not re-admitted to the hospital.

At 206, the key features used in the treatment process of other patients for the same medical condition of the patient are identified. The pre-authorization program 110a, 110b may analyze similarities between the current patient and the cohorts that may have had a positive outcome by factors or attributes, such as age, gender, race, family medical history, use of tobacco, use of alcohol and by profession using NLP and ML algorithms. The pre-authorization system 110a, 110b may identify prescribed medications, lab results and examination results based on the EHR data of the patients. Different features may be associated with different medical conditions (e.g., different diseases) and the medical conditions may be extracted from and existing credible medical database and medical literature. For example, key features for a neurosurgery patient are age, gender, race, family medical history, use of tobacco, use of alcohol, CT results and MRI results.

At 208, the closest features that can be applied to the patient are identified. The closest features may include features that are most relevant to the particular disease the current patient has or is exhibiting. The closest features may be identified based on the analysis conducted and identified in steps 202, 204 and 206. Features identified may, for example, be the most impactful features identified as similar features that were identified in causing positive outcomes for many cohorts of the current patient. Pairing the identified impactful features for a particular surgery with the closest features that the patient exhibits, such as age, weight, profession and symptoms may provide optimal features to treat the disease.

Then, at 210, the stakeholders are identified based on the key features and the closest identified features. The stakeholders may include people who are relevant to the medical procedure of the patient and the key features, such as doctors and medical networks that provide care for a particular disease within the medical insurance policy of the current patient and the location, country or state that the current patient resides or is traveling to. For example, ML and NLP may analyze an insurance policy, a hospital policy and government policy to identify and stakeholders who may offer approval for the recommended medical procedures for the current patient. The stakeholder may, for example, have a specialty in certain medications or a specialty in reading CT scans or MRI scans, such as a radiologist. Depending on the recommended procedure for the patient, many stakeholders may be required to authorize each step leading up to, for example, a surgery and stakeholders may also be required to authorize a recovery plan for the patient.

ML and NLP may analyze the key features, the patient data, the patient location, the patient medical insurance policy and governmental regulations to identify which stakeholders may be involved in the approval process for the patient. The analysis may provide, for example, the step by step procedures and timelines of approvals for pre- and post-operation examinations, tests, primary care doctor visits, specialist doctor visits and surgical care appointments such that the timeline may be reduced between each visit, which impacts the patient's recovery and overall health by reducing the wait time leading up to surgery and post-surgery care.

At 212, the stakeholders are authorized, and new blocks are created. The stakeholders identified at step 210 may be authorized to provide pre-authorization for patient care. Pre-authorization may, for example, preempt care for a patient during the beginning of the medical procedure process. The stakeholders may be added to the blockchain processing system. Each request to the stakeholder may be considered a block and the block may be created by the pre-authorization program 110a 110b. The corresponding transaction (i.e., pre-authorization) data that is recorded in the system may be called blocks. The blocks are created in the existing blockchain framework, such as Hyperledger. There may be different blockchain frameworks available. A different framework may be used based on the business use case needs. At a high level, the blocks creation and appending the blocks to the chain may still be handled by these frameworks.

At 214, the new blocks are added to the blockchain for processing stakeholder authorizations. The blocks are appended to the blockchain using existing blockchain frameworks, such as Hyperledger.

It may be appreciated that FIG. 2 provides only an illustration of one embodiment and do not imply any limitations with regard to how different embodiments may be implemented. Many modifications to the depicted embodiment(s) may be made based on design and implementation requirements.

FIG. 3 is a block diagram 900 of internal and external components of computers depicted in FIG. 1 in accordance with an illustrative embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902a, b and external components 904a, b illustrated in FIG. 3. Each of the sets of internal components 902a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the pre-authorization program 110a in client computer 102, and the pre-authorization program 110b in network server 112, may be stored on one or more computer-readable tangible storage devices 916 for execution by one or more processors 906 via one or more RAMs 908 (which typically include cache memory). In the embodiment illustrated in FIG. 3, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the pre-authorization program 110a, 110b can be stored on one or more of the respective portable computer-readable tangible storage devices 920, read via the respective R/W drive or interface 918 and loaded into the respective hard drive 916.

Each set of internal components 902a, b may also include network adapters (or switch port cards) or interfaces 922 such as a TCP/IP adapter cards, wireless wi-fi interface cards, or 3G or 4G wireless interface cards or other wired or wireless communication links. The software program 108 and the pre-authorization program 110a in client computer 102 and the pre-authorization program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the pre-authorization program 110a in client computer 102 and the pre-authorization program 110b in network server computer 112 are loaded into the respective hard drive 916. The network may comprise copper wires, optical fibers, wireless transmission, routers, firewalls, switches, gateway computers and/or edge servers.

Each of the sets of external components 904a, b can include a computer display monitor 924, a keyboard 926, and a computer mouse 928. External components 904a, b can also include touch screens, virtual keyboards, touch pads, pointing devices, and other human interface devices. Each of the sets of internal components 902a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Analytics as a Service (AaaS): the capability provided to the consumer is to use web-based or cloud-based networks (i.e., infrastructure) to access an analytics platform. Analytics platforms may include access to analytics software resources or may include access to relevant databases, corpora, servers, operating systems or storage. The consumer does not manage or control the underlying web-based or cloud-based infrastructure including databases, corpora, servers, operating systems or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

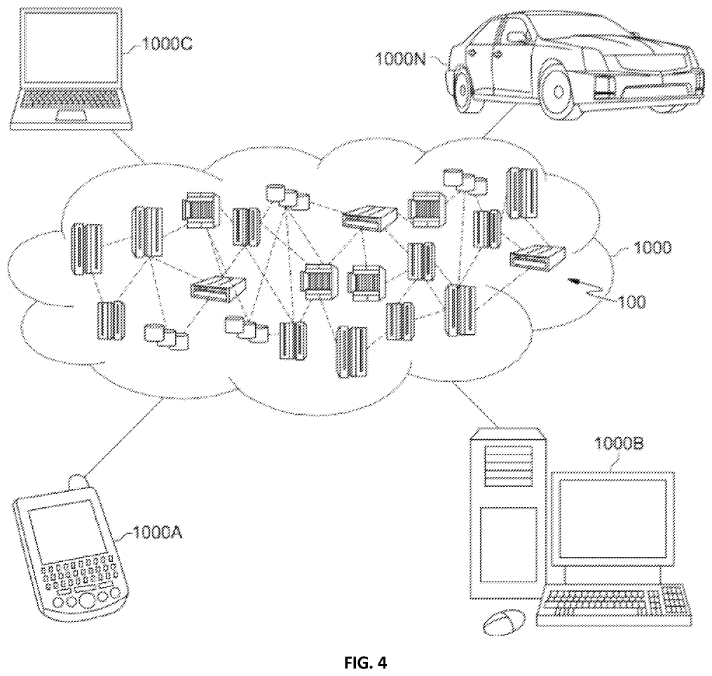

Referring now to FIG. 4, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 4 are intended to be illustrative only and that computing nodes 100 and cloud computing environment 1000 can communicate with any type of computerized device over any type of network and/or network addressable connection (e.g., using a web browser).

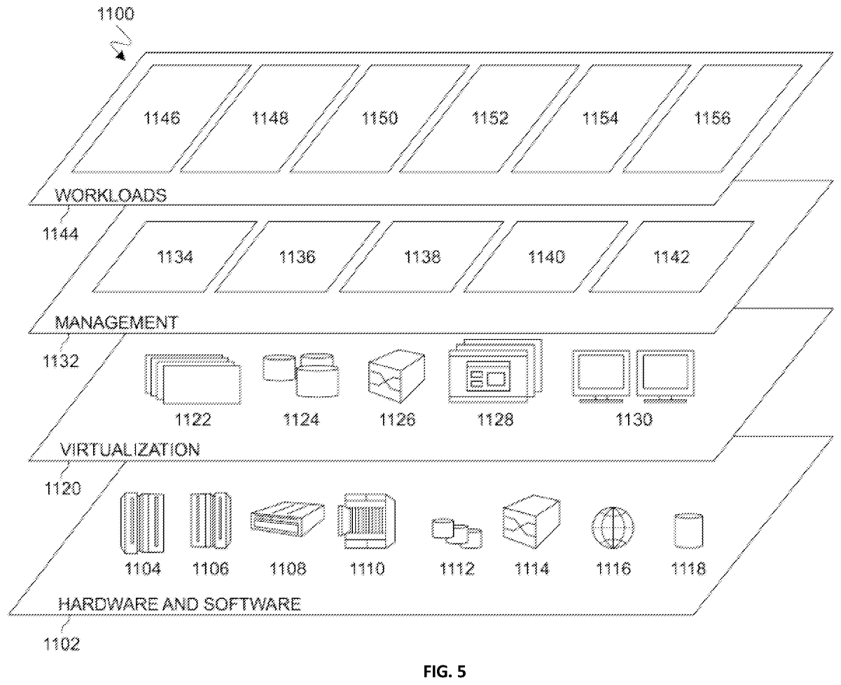

Referring now to FIG. 5, a set of functional abstraction layers 1100 provided by cloud computing environment 1000 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and pre-authorization 1156. A pre-authorization program 110a, 110b provides a way to reduce patient wait times by using NLP and ML combined with key features of successful treatments to identify key stakeholders for authorization.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer implemented method for pre-authorization, the method comprising:
identifying a treatment for a medical condition that yields a positive outcome;
identifying a plurality of key features in the identified treatment;
identifying a plurality of features within the plurality of key features that can be applied to a patient;
identifying a stakeholder based on the identified plurality of features applied to the patient;
authorizing the identified stakeholder;
creating a new block based on the authorized stakeholder; and
adding the new block to a blockchain network for processing authorizations.

2. The method of claim 1, further comprising:
analyzing the medical condition of the patient; and
providing authorization to the patient for the treatment of a medical condition.

3. The method of claim 1, further comprising:
analyzing the medical condition of the patient; and
providing authorization to the patient for the treatment of a medical condition using a smart contract.

4. The method of claim 1, wherein the plurality of key features are features related to similar features that yielded positive outcomes for a medical condition.

5. The method of claim 1, wherein the plurality of features are identified through natural language processing (NLP), wherein the plurality of features are stored on an electronic health record (EHR) of the patient.

6. The method of claim 1, wherein the stakeholders include individuals authorized to further the medical care of the patient.

7. The method of claim 3, wherein the smart contract has a list of authorizers (stakeholders) associated with the medical condition and the medical treatment.

8. A computer system for pre-authorization, comprising:
one or more processors, one or more computer-readable memories, one or more computer-readable tangible storage media, and program instructions stored on at least one of the one or more computer-readable tangible storage media for execution by at least one of the one or more processors via at least one of the one or more computer-readable memories, wherein the computer system is capable of performing a method comprising:
identifying a treatment for a medical condition that yields a positive outcome;
identifying a plurality of key features in the identified treatment;
identifying a plurality of features within the plurality of key features that can be applied to a patient;
identifying a stakeholder based on the identified plurality of features applied to the patient;
authorizing the identified stakeholder;
creating a new block based on the authorized stakeholder; and
adding the new block to a blockchain network for processing authorizations.

9. The computer system of claim 8, further comprising:
analyzing the medical condition of the patient; and
providing authorization to the patient for the treatment of a medical condition.

10. The computer system of claim 8, further comprising:
analyzing the medical condition of the patient; and
providing authorization to the patient for the treatment of a medical condition using a smart contract.

11. The computer system of claim 8, wherein the plurality of key features are features related to similar features that yielded positive outcomes for a medical condition.

12. The computer system of claim 8, wherein the plurality of features are identified through natural language processing (NLP), wherein the plurality of features are stored on an electronic health record (EHR) of the patient.

13. The computer system of claim 8, wherein the stakeholders include individuals authorized to further the medical care of the patient.

14. The computer system of claim 10, wherein the smart contract has a list of authorizers (stakeholders) associated with the medical condition and the medical treatment.

15. A computer program product for pre-authorization, comprising:
one or more computer-readable tangible storage media and program instructions stored on at least one of the one or more computer-readable tangible storage media, the program instructions executable by a processor to cause the processor to perform a method comprising:
identifying a treatment for a medical condition that yields a positive outcome;
identifying a plurality of key features in the identified treatment;
identifying a plurality of features within the plurality of key features that can be applied to a patient;
identifying a stakeholder based on the identified plurality of features applied to the patient;
authorizing the identified stakeholder;
creating a new block based on the authorized stakeholder; and
adding the new block to a blockchain network for processing authorizations.

16. The computer program product of claim 15, further comprising:
analyzing the medical condition of the patient; and
providing authorization to the patient for the treatment of a medical condition.

17. The computer program product of claim 15, further comprising:
analyzing the medical condition of the patient; and
providing authorization to the patient for the treatment of a medical condition using a smart contract.

18. The computer program product of claim 15, wherein the plurality of key features are features related to similar features that yielded positive outcomes for a medical condition.

19. The computer program product of claim 15, wherein the plurality of features are identified through natural language processing (NLP), wherein the plurality of features are stored on an electronic health record (EHR) of the patient.

20. The computer program product of claim 15, wherein the stakeholders include individuals authorized to further the medical care of the patient.
